(19) United States
(12) Patent Application Publication
(10) Pub. No.: US 2010/0312128 A1
(43) Pub. Date: Dec. 9, 2010

(54) SYSTEMS AND METHODS FOR MONITORING BLOOD PARTITIONING AND ORGAN FUNCTION

(76) Inventors: Edward Karst, S. Pasadena, CA (US); Brian Jeffrey Wenzel, San Jose, CA (US); Allen J. Keel, San Francisco, CA (US); Wenbo Hou, Lancaster, CA (US); Taraneh Ghaffari Farazi, San Jose, CA (US)

Correspondence Address: STEVEN M MITCHELL;PACESETTER INC, 701 EAST EVELYN AVENUE, SUNNYVALE, CA, 94086, US

(21) Appl. No.: 12/648,595

(22) Filed: Dec. 29, 2009

Related U.S. Application Data
(60) Provisional application No. 61/185,520, filed on Jun. 9, 2009.

(51) Int. Cl. A61B 5/0265

(52) U.S. Cl. 600/506; 600/504

(57) ABSTRACT

Methods and systems for monitoring an organ of interest within a patient use one or more sensors to obtain one or more signals indicative of one or more of blood being provided to the organ of interest, blood being received from the organ of interest, and blood present in the organ of interest. Changes in an amount of blood being provided to the organ of interest, an amount of blood being received from the organ of interest, and/or an amount of blood present in the organ of interest are monitored based on changes in the obtained signal(s). Such methods and systems can be used to detect dysfunction of the organ of interest or tumor growth in the organ of interest, but are not limited thereto.

### PRIORITY CLAIM

This application claims priority under 35 U.S.C. 119(e) to U.S. Provisional Patent Application No. 61/185,520, entitled SYSTEMS AND METHODS FOR MONITORING BLOOD PARTITIONING AND ORGAN FUNCTION, filed Jun. 9, 2009 (Attorney Docket No. A09P3010), which is incorporated herein by reference.

### FIELD OF THE INVENTION

Embodiments of the present invention relate to implantable systems that are useful for obtaining measurements of blood volume provided to, received from, or present in a vital or non-vital organ of interest, and methods for use therewith.

### BACKGROUND OF THE INVENTION

An organ in a human body can become dysfunctional and/or fail due to a variety of reasons, for example in response to trauma, medications that may be toxic to the organ, hematological malignancies, disease, tumor growth, sepsis and other causes of tissue inflammation, and other conditions.

The onset of organ dysfunction and/or organ failure can be acute or chronic, and for some organs can require medical intervention to restore homeostasis of the circulatory system. Problematically, early symptoms of organ dysfunction and/or failure may be fairly general and may or may not provide sufficient warning to a patient of potential problems. For example, early symptoms such as those of slower onset chronic kidney failure may include fatigue and listlessness which symptoms are common to a variety of illnesses.

Once symptoms are identified, a physician or medical technician can perform a diagnostic workup to determine the cause of such symptoms. Such a diagnostic workup can include myriad different diagnostic tests, and may include tests unnecessary and/or unrelated to the organ of interest. For example, in the case of suspected kidney dysfunction a diagnostic workup can include some or all of a complete blood count (CBC), urinalysis, urine culture and colony count, serum and urine osmolality, chemistry panel, sedimentation rate, arterial blood gas analysis, blood volume, cystoscopy and retrograde pyelography, a nephrology consult, and a urology consult. Additional studies include abdominal CT scans, ultrasonography, and a renal biopsy.

Further, some organ dysfunction and/or failure related to tumor growth may not cause visible symptoms to appear until tumor growth has reached a later, less treatable stage. For example, the five-year survival rate for pancreatic cancer is low, at 5 percent, because pancreatic cancer is often not diagnosed until its later stages. There is no test for early detection of pancreatic cancer, and such symptoms as weight loss and abdominal discomfort are often mild.

It would be desirable to have and apply a diagnostic technique before and while an organ of interest begins to fail or suffer dysfunction so that medical intervention can be performed more quickly and correctly to the organ of interest. Further, it would be desirable to have and apply a diagnostic technique before and while an organ of interest begins to fail or suffer dysfunction so that a more narrowly targeted diagnostic workup can be performed soon after or before commonly visible symptoms appear.

### SUMMARY OF THE INVENTION

Embodiments of the present invention relate to systems and methods that are useful for obtaining measurements of blood volume provided to, received from, or present in a vital and/or non-vital organ of interest. Such systems and methods can be used to monitor an organ of interest, such as a kidney, for the purpose of detecting organ function (e.g., organ dysfunction), or growth of a tumor within the organ, but is not limited thereto. Many such embodiments are directed to chronically implantable systems, and methods for use therewith.

In accordance with an embodiment, one or more sensors is/are used to obtain one or more signals (e.g., plethysmography signals) indicative of blood being provided to the organ of interest, blood being received from the organ of interest, and/or blood present in the organ of interest. The one or more sensors can, e.g., each be an impedance sensor including at least two electrodes, or an optical sensor including a light source and a light detector, but are not limited thereto. Based on changes in at least one of the obtained signal(s), changes in an amount of blood being provided to the organ of interest, an amount of blood being received from the organ of interest, and/or an amount of blood present in the organ of interest are monitored. This can include determining, from time to time, one or more metrics based on the obtained signal(s), wherein the one or more metrics is/are indicative of blood being provided to the organ of interest, blood being received from the organ of interest, and/or blood present in the organ of interest. Changes in the determined metric(s) over time are monitored to thereby monitor changes an amount of blood being provided to the organ of interest, an amount of blood being received from the organ of interest, and/or an amount of blood present in the organ of interest. In specific embodiments, such monitoring includes monitoring whether the amount of blood being provided to the organ of interest, the amount of blood being received from the organ of interest, and/or the amount of blood present in the organ of interest is increasing, decreasing or staying relatively the same.

In accordance with an embodiment, an alert and/or therapy can be trigged based on the results of the monitoring. This can include comparing a determined metric to a corresponding baseline, and triggering an alert and/or therapy if the metric falls below or rises above the corresponding baseline by at least a specified threshold. Such metrics can be indicative of blood volume of blood vessels known to provide blood to the organ of interest, or indicative of venous oxygen saturation or arterial oxygen saturation. Such metrics can be determined, e.g., based on a peak-to-peak amplitude, an area under the curve, a full width at have max, and/or a downward slope of after a peak amplitude of one or more obtained signals.

Where the organ of interest is a kidney, at least one sensor can be used to obtain a signal indicative of renal blood flow being provided to the kidney, and changes the renal blood flow can be monitored based on monitored changes over time in the obtained signal. For a specific example, a sensor can be used to obtain a plethysmography signal indicative of changes in blood volume of glomerular capillaries, renal arteries or renal veins. Such an embodiments can be used, e.g., to detect kidney disfunction based on comparisons of a metric of the obtained signal to a baseline and/or threshold.

In accordance with an embodiment, a sensor is implanted extravascularly within the patient at a location adjacent to the organ of interest or one or more blood vessels that provide blood to or receive blood from the organ of interest. In an alternative embodiment, a non-implanted sensor is located against the patient's skin at a location adjacent to one or more blood vessels that provide blood to or receive blood from the organ of interest.

In some embodiments, an alert and/or therapy can be triggered, e.g., if the monitored changes are indicative of tumor growth in the organ of interest, or dysfunction of the organ of interest.

Certain embodiments are directed to methods and systems for monitoring for sepsis. One or more sensors is/are used to obtain signal(s) indicative of blood being provided to a vital organ, blood being received from the vital organ, and/or blood present in the vital organ. Additionally, one or more further sensors is/are used to obtain signal(s) indicative of blood being provided to a non-vital organ, blood being received from the non-vital organ, and/or blood present in the non-vital organ. Monitoring for sepsis is performed based on a comparison between the signal(s) indicative of blood to, from and/or in the vital organ and the signal(s) indicative of blood to, from and/or in the non-vital organ. This can include determining one or more metrics of signal(s) indicative of blood being provided to the vital organ, blood being received from the vital organ, and/or blood present in the vital organ, and determining corresponding metrics for the non-vital organ. Such determined metrics can then be compared to determine whether sepsis is occurring, e.g., if the comparisons of the metrics are indicative of increasing blood flow to the vital organs and reducing blood flow to the non-vital organ. An alert and/or therapy can be triggered if sepsis is detected.

Additional and alternative embodiments, features and advantages of the invention will appear from the following description in which the preferred embodiments have been set forth in detail, in conjunction with the accompanying drawings and claims.

### BRIEF DESCRIPTION OF THE FIGURES

FIG. 1A is a diagram of the human circulatory system.

FIG. 1B is an exemplary photoplethysmograph (PPG) spanning two cardiac cycles.

FIG. 2A is a diagram of an embodiment of a system and method of monitoring blood perfusion to an organ of interest in accordance with the present invention.

FIG. 2B is a photoplethysmograph illustrating one technique for determining a metric of blood flow.

FIG. 2C is a photoplethysmograph illustrating an alternative technique for determining a metric of blood flow.

FIG. 2D is a photoplethysmograph illustrating a further technique for determining a metric of blood flow.

FIG. 3A is a diagram of an alternative embodiment of a system and method of monitoring blood perfusion to an organ of interest in accordance with the present invention comprising an implantable probe.

FIG. 3B is a detailed side view of the implantable probe of FIG. 3A.

FIG. 3C is a cross-section of the implantable probe of FIG. 3B.

FIG. 4 is a diagram of the system of FIG. 3A positioned to monitor blood perfusion to the liver

FIG. 5 is a diagram of an embodiment of a system and method of monitoring blood perfusion to an organ of interest in accordance with the present invention comprising an implantable probe wireless connected with an implant device.

FIG. 6 is a diagram of an embodiment of a system and method of monitoring blood perfusion to an organ of interest in accordance with the present invention comprising an implant device providing a dedicated optical sensor.

FIG. 7 is a diagram of a further embodiment of a system and method of monitoring blood perfusion to an organ of interest in accordance with the present invention comprising an implantable probe.

FIG. 8A is a diagram of an embodiment of a system and method of monitoring blood perfusion to an organ of interest in accordance with the present invention comprising multiple implantable probes.

FIG. 8B is a flowchart of the method of monitoring blood perfusion of FIG. 8A.

FIG. 9 is a diagram of an embodiment of a system and method of monitoring blood perfusion to a vital and a non-vital organ to detect systemic dysfunction.

FIG. 10A illustrates an exemplary implantable stimulation device that includes a PPG sensor, and which can be used to perform embodiments of the present invention.

FIG. 10B is a simplified block diagram that illustrates possible components of the implant device shown in FIG. 10A.

### DETAILED DESCRIPTION OF THE INVENTION

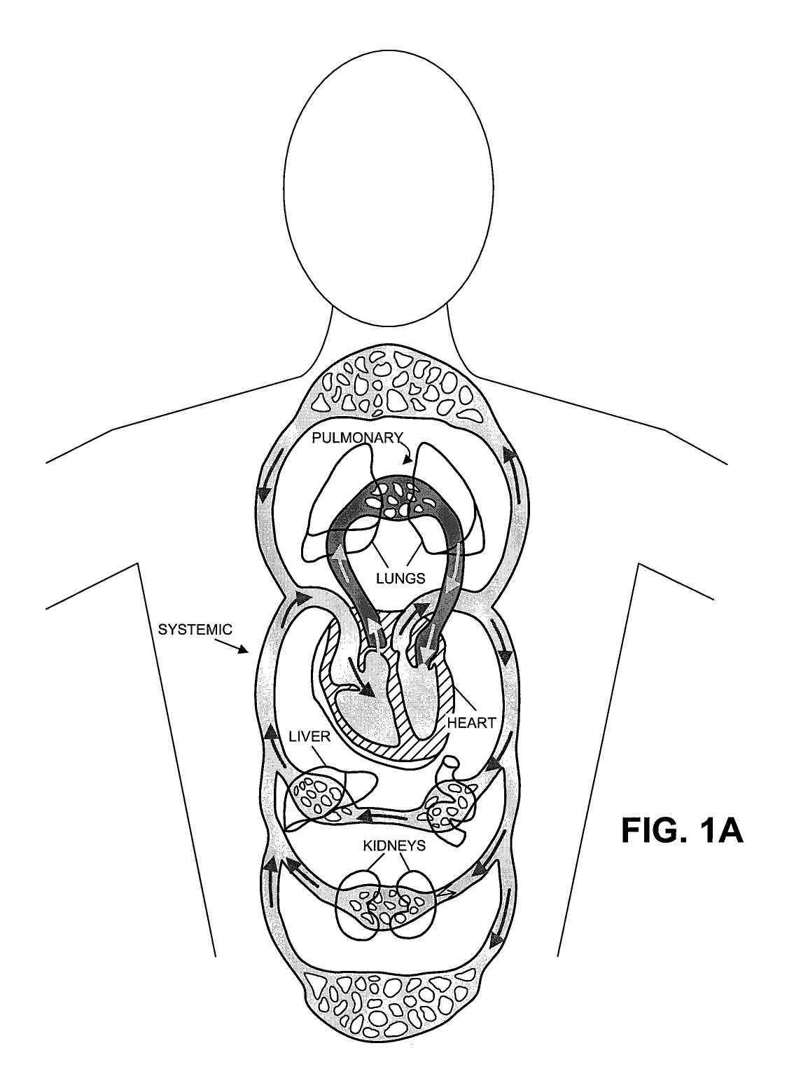

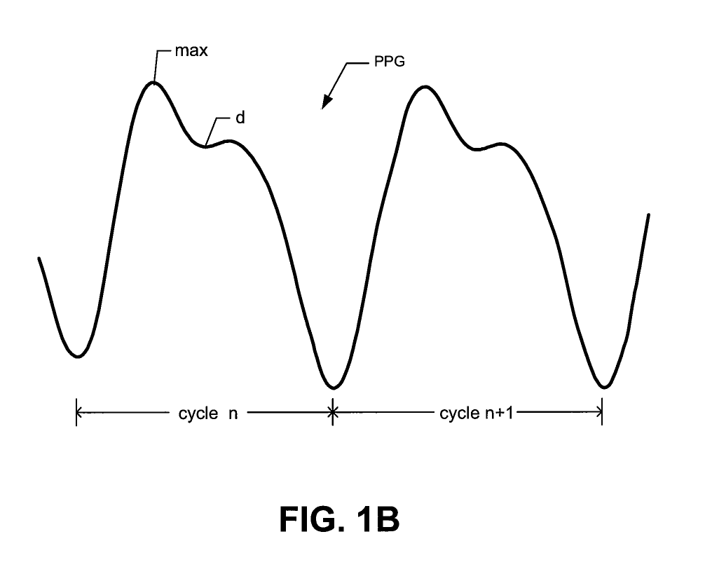

Referring to FIG. 1, the human circulatory system includes both systemic and pulmonary circulation systems. The heart serves as a pump that maintains blood circulation. The pulmonary circulation system (darkly shaded path) supplies the lungs with blood flow, while the systemic circulation system (lightly shaded path) supplies blood to other parts of the body including the other vital organs (e.g., liver, kidneys) and non-vital organs (e.g., spleen) of the body. Both the pulmonary and systemic circulatory systems are made up of arteries, arterioles, capillaries, venules and veins. The arteries take the blood from the heart, while the veins return the blood to the heart.

Blood flow characteristics can vary with organ performance, and abnormalities in characteristics of blood circulation can be symptomatic of dysfunction and/or failure of the organ. Monitoring blood perfusion to a specific organ of interest can allow detection of abnormalities in characteristics of blood circulation. Characteristics of blood circulation can include the amount of blood provided to an organ, the amount of blood received from an organ, and the amount of blood present in the organ.

One technique for measuring blood flow—by way of blood volume in tissue—is plethysmography. Photoplethysmography is an optical technique that uses an optical sensor to illuminate tissue and measure changes in light absorption. A pulse oximeter is an example of one type of optical sensor typically placed at a finger tip to measure transmissive absorption or against the forehead to measure reflective absorption. A conventional pulse oximeter monitors the perfusion of blood to the dermis and subcutaneous tissue of the skin. The heart pumps blood to the periphery of the body with each cardiac cycle. The pressure pulse is somewhat damped by the time it reaches the skin but is sufficient to distend the arteries and arterioles in the subcutaneous tissue. If the pulse oximeter is attached without compressing the skin, a pressure pulse can also be seen from the venous plexus, as a small secondary peak. An example of a signal generated by an optical sensor is shown in FIG. 1B as a photoplethysmograph (PPG) comprising multiple cardiac cycles (n, n+1). Each cardiac cycle appears as a peak (max) in the PPG signal. The DC component of the PPG signal is attributable to the bulk absorption of the skin tissue, while the AC component is directly attributable to variation in blood volume in the skin caused by the pressure pulse of the cardiac cycle. A PPG signal (also referred to herein simply as a PPG) is generally processed to determine heart rate; however, blood flow to the skin can be modulated by multiple other physiological systems, and the PPG signal is also used to monitor breathing, hypovolemia, and other systemic signs.

Embodiments of systems and methods in accordance with the present invention can comprise optical sensors that respond to pulsations and can provide information on perfusion of an individual organ by targeting structures of the circulatory system that provide blood to or receive blood from the organ of interest. Perfusion measurement to the individual organ can enable detection of organ health, apart from overall systemic health. For example, a change in amplitude of a PPG signal obtained using an optical sensor positioned to probe renal blood flow could indicate disruption in renal functions. Signals obtained by the optical sensor can be monitored to detect changes in one or more characteristics of blood circulation related to the organ of interest.

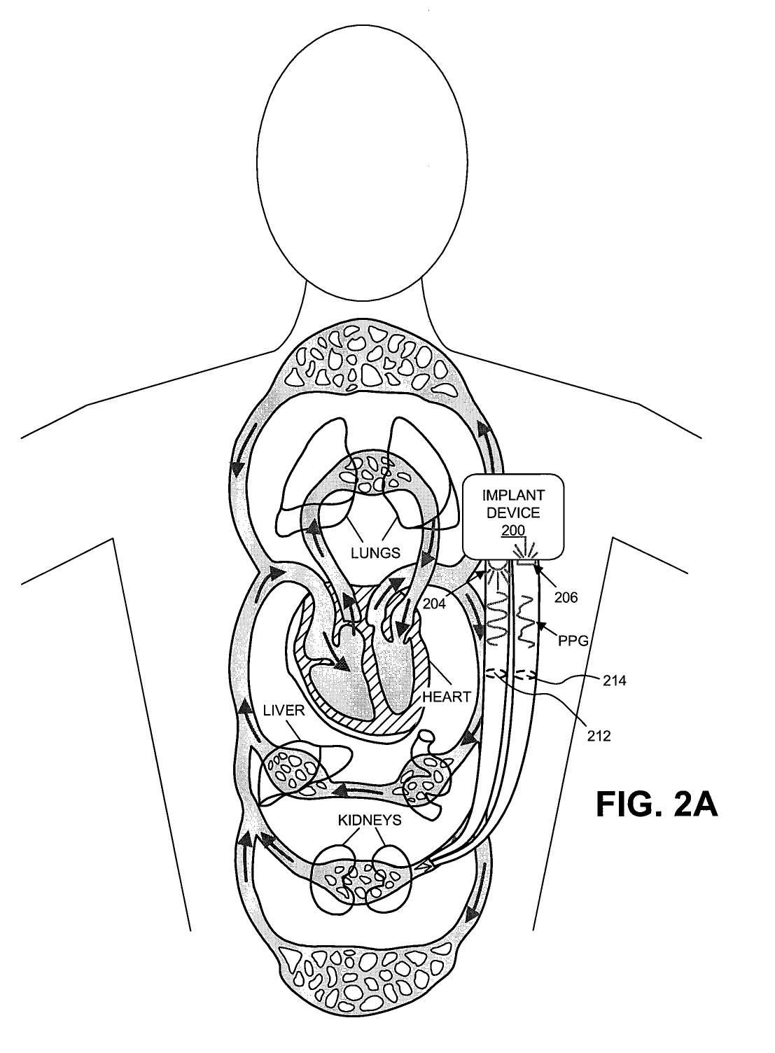

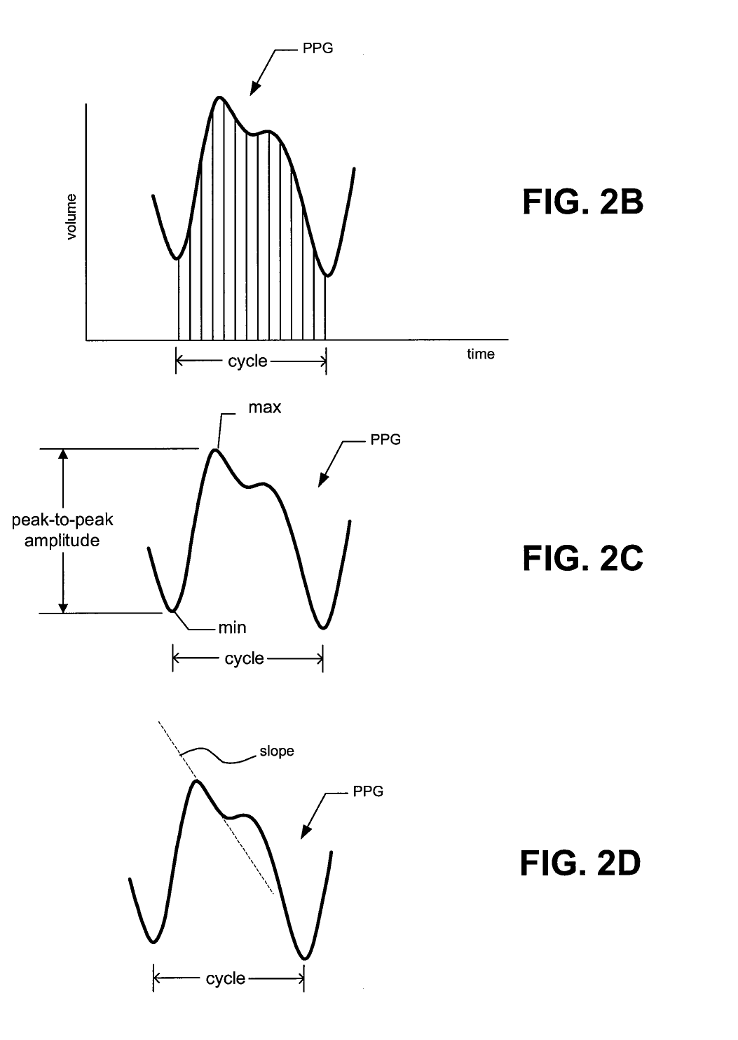

Referring to FIG. 2A an embodiment of a system and method of monitoring blood perfusion to an organ of interest in accordance with the present invention is shown. The system comprises an implant device 200 including an optical sensor resident in or otherwise operably connected with the implant device 200. The implant device 200 can function in a dedicated capacity with the optical sensor, or alternatively the implant device 200 can perform multiple different functions. For example, the implant device 200 may include an optical sensor and an artificial pacemaker and/or an implantable cardioverter-defibrillator (ICD), with some or no shared electronic circuitry and/or energy source. The site of implantation within a patient's body can be determined based on the functions performed by the implant device 200 and/or physiological preferences. For example, if the implant device 200 provides pacing for the patient, the implant device 200 can be implanted within a chest cavity of the patient, as shown in FIG. 2A (note that the organs including the heart are displaced and resized within the diagram to better illustrate the separate circulatory systems).

A single or multi-wavelength light source 204 of the optical sensor resident in the implant device 200 or otherwise operably connected with the implant device 200 can illuminate a target. The light source 204 can include one or more light emitted diodes (LEDs), laser diodes, organic light emitting diodes (OLEDs), liquid crystal display (LCD), bulbs or other light emitting structures. A multi-wavelength light source can include multiple light emitting devices each device emitting light at different wavelengths. A first fiber optic guide 212 can direct light emitted by the light source 204 to the target, combining the multiple wavelengths into a single beam. A second fiber optic guide 214 can direct light reflected or transmitted by the target to a light detector 206 resident in the implant device 200 or otherwise associated with the implant device 200. The light detector 206 can include one or more photo-detectors and/or photo-resistors, or other structure for detecting reflected or absorbed light. In an embodiment, the fiber optic guides 212, 214 can be fixedly connected so that the fiber optic guides 212, 214 are predictably oriented with respect to one another. The fiber optic guides 212, 214 are preferably routed subcutaneously. Alternatively, the fiber optic guides 212, 214 can be routed to exit the body and reenter the body at a position advantage to reaching the target. Such an arrangement, while possible, may not be practical due to a risk infection and/or dislocation.

In the exemplary embodiment shown in FIG. 2A, the target is a renal artery providing blood to a kidney. The light source emits light at one or more wavelengths and the light detector measures the light absorbed or reflected over multiple cardiac cycles to generate one or more PPG signals indicative of a volume of blood present in the renal artery. A metric can be determined based on the one or more PPG signals. For example, the metric can be calculated as an area under the curve of one or more cardiac cycles of the one or more PPG signals, as shown in FIG. 2B. Alternatively, the metric can be calculated as the peak-to-peak amplitude of the one or more PPG signals, as shown in FIG. 2C. Alternatively, the metric can be determined from some other feature of the one or more PPG signals. For example, the metric can be derived from a downward slope after reaching a peak amplitude, as shown in FIG. 2D, which metric can be indicative of blood volume.

Implant devices for use with systems and methods in accordance with the present invention can include circuitry to receive the one or more PPG signals from the optical sensor and determine a metric based on the one or more PPG signals to monitor blood volume provided to or received from the organ of interest. Alternatively, the implant device can determine a metric based on the one or more PPG signals and communicate the metric to an external computer for monitoring by the external computer. Alternatively, the implant device can serve as a buffer that collects the one or more PPG signals and communicates the PPG one or more signals to an external device, which external device determines a metric and monitors the metric.

The metric can be monitored to detect changes in the metric which can be indicative of organ dysfunction and/or organ failure. For example, if the metric is a downward slope as shown in FIG. 2D, if the downward slope is relatively shallow when compared with an established baseline, the metric can indicate a high volume of blood that is necessarily pushed out from the organ over a relatively long period of time. Contrariwise, if the downward slope is relatively steep when compared with the established baseline, the metric can indicate a low volume of blood that is quickly pushed out from the organ. A drop in blood volume provided to the organ can be an indication of an ischemic condition. Ischemia can cause tissue to become hypoxic, or, if no oxygen is supplied at all, anoxic. This can cause ischemic cell death. lschemia is a feature of heart diseases, transient ischemic attacks, cerebrovascular accidents, ruptured arteriovenous malformations, and peripheral artery occlusive disease. The heart and kidneys are among the organs that are the most sensitive to ischemia. A physician may desire to monitor one or more organs of a patient for a patient known to be at risk of a physiological disorder that can cause dysfunction and/or failure in the one or more organs.

An organ of interest can be identified preventatively through personal medical history, family medical history, and/or DNA profile, or an organ of interest can be identified as part of a treatment plan. For example, in an embodiment of a system and method of monitoring for tumor damage and/or growth in accordance with the present invention, an optical sensor can be positioned to measure blood volume provided to an organ from which a tumor is removed, the organ being in remission. Blood perfusion to the organ can be monitored, as described above. Damage caused by the tumor or removal of the tumor may be cause the organ to be dysfunctional and/or at risk of failure. Further, an increase of blood volume provided to the organ can indicate a recurrence of tumor growth. Such systems and methods can be useful for early detection and treatment. For example, where positioned at a pancreas or lung, early detection of tumor growth can greatly improve chances of survival.

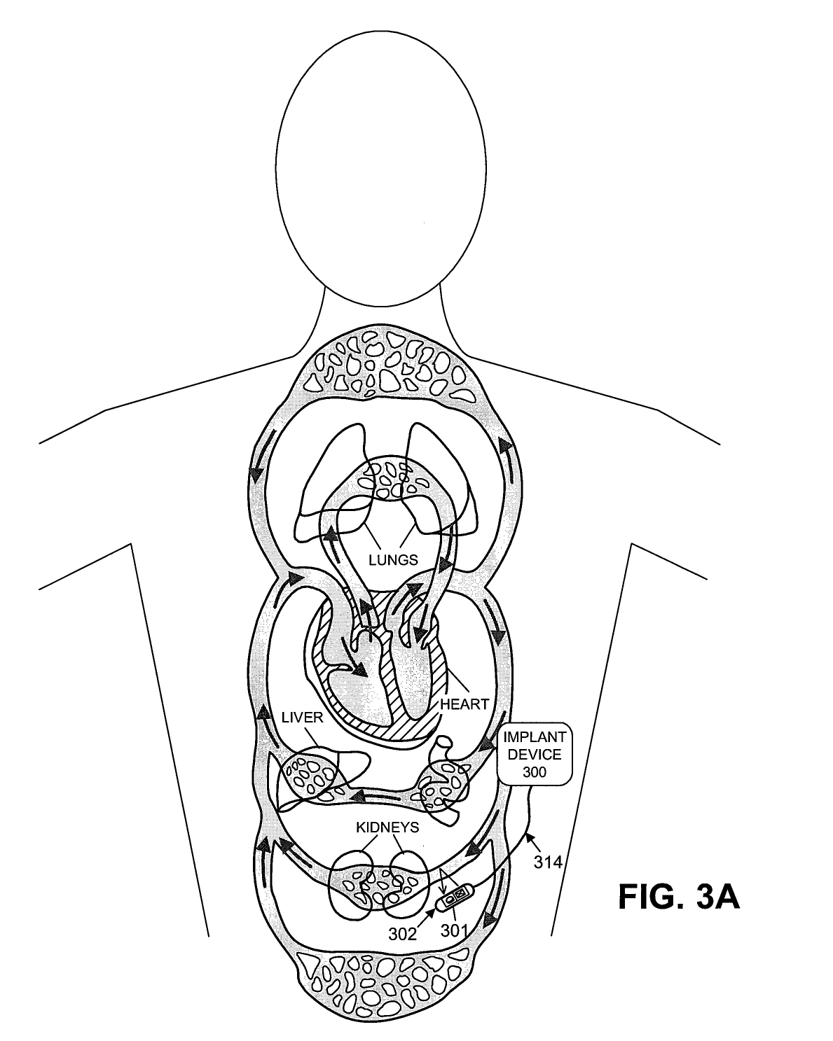

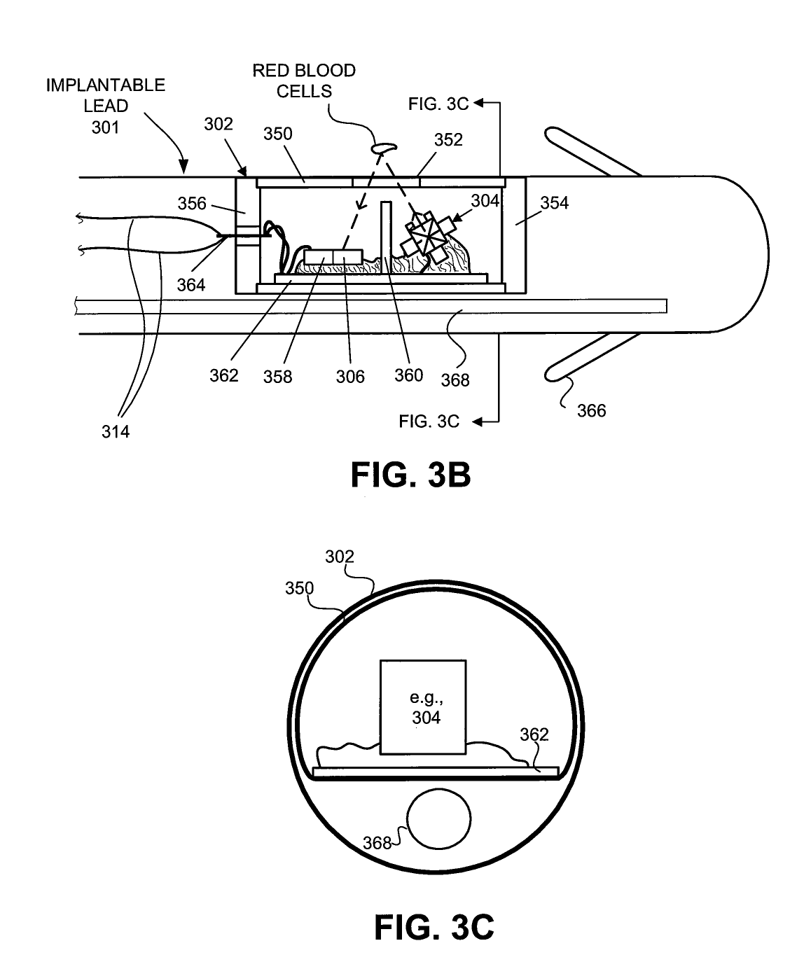

Referring to FIG. 3A, an alternative embodiment of a system and method of monitoring blood perfusion to an organ of interest in accordance with the present invention is shown. The system comprises an optical sensor 302 housed in an implantable probe 301, e.g., resembling the implantable leads described in detail in U.S. Ser. No. 11/231,555 entitled “IMPROVED MULTI-WAVELENGTH IMPLANTABLE OXIMETER SENSOR,” incorporated herein by reference. The optical sensor 302 is operably connected with an implant device 300 by one or more wires 314. As above, the implant device 300 can function in a dedicated capacity with the optical sensor 302, or alternatively the implant device 300 can perform multiple different functions. As shown, the implant device 300 is a dedicated device that processes signals from the optical sensor 302. The implant device 300 is preferably positioned subcutaneously near a site of monitoring to reduce invasiveness of the system.

As above, optical sensors for use with embodiments of systems in accordance with the present invention can comprise a single or multi-wavelength light source and one or more photo-detectors and/or photo-resistors. Referring to FIG. 3B, a light source 304 and light detector 306 can be built into an optical sensor 302, the optical sensor 302 being fixable to and/or within the implantable probe 301. As described in detail in U.S. Ser. No. 11/231,555, the light source can be a multi-wavelength light source relying on a beam combiner 304 to permit multiple wavelengths of emitted light to be combined into a single beam. The optical sensor 302 includes a housing within which are components including the beam combiner 304, a light detector 306 and optionally an application specific integrated circuit (ASIC) 358. The housing can comprise a tube 350 and a pair of end caps 354 and 356 that can be used to hermetically seal the components within the housing. The tube 350 can be made of an opaque material, such as metal (e.g., titanium or stainless steel) or ceramic, so long as it includes a window 352 that passes light of all the wavelengths of interest in the combined light beam. In an alternative embodiment, the entire tube 350 can be made of a material that passes light of all the wavelengths of interest in the combined beam, and thus, in this embodiment the entire tube 350 can be considered a window. Further, the portion of the probe 301 that is adjacent to the window 352 of the optical sensor 302, where light is to exit and enter, should allow the light to pass in and out of the optical sensor 302. Thus, the probe 301 may be transparent, or include a window, opening, or the like.

The beam combiner 304, the window 352 and the light detector 306 should be positioned such that the combined light beam produced by the beam combiner 304 exits the housing through the window 352 and such that the light backscattered from blood (outside the window) will be scattered back toward the photo detector 306. An opaque optical wall 360 is positioned between the beam combiner 304 and the light detector 306, so that light is not internally reflected from the beam combiner 304 to the light detector 306. Where present, the ASIC 358, which can include filters, analog-to-digital circuitry, multiplexing circuitry, and the like, controls the light source 304 and processes the light detector signals produced by the light detector 306 in any manner well known in the art. The ASIC 358 preferably provides digital signals indicative of the light detector signals to the implant device 300. If an ASIC 358 or equivalent circuitry is not included within the optical sensor 302, analog signals can be delivered between the optical sensor 302 and the implant device 300. The beam combiner 304, optical wall 360, light detector 306 and ASIC 358 can be attached to a substrate 362, e.g., by an epoxy. The substrate can be, e.g., a printed circuit board (PCB). Bond wires can be used to attach the various components to the substrate 362, as well as to attach the substrate 362 to feedthroughs attached to wires 314 connecting the optical sensor 302 to the implant device 300.

The implantable probe 301 is shown as including tines 366 for attaching the probe in a desired position, but may include any other type of fixation technique or none at all. Additionally, the implantable probe 301 may also include a lumen 368 for a stylet, which can be used for guiding the probe to its desired position. Wires 314 provide power and optionally control signals to the optical sensor 302 from the implant device 300, and provide PPG and/or pulse oximetry signals from the sensor 302 to the implant device 300. Referring to FIG. 3C, the tube 350 is generally “D” shaped, so that it can be readily included within the implantable probe 301 while still allowing the lumen 368 to fit within the same inner-space of the implantable probe 301. Alternative shapes are also within the scope of the present invention.

Referring again to FIG. 3A, the implantable probe 301 can be positioned to target structures of the circulatory system that provide blood or receive blood from the organ of interest. As shown, the implantable probe 301 is positioned so that the optical sensor 302 emits and receives light from a renal artery carrying blood to a kidney. The light source emits light at one or more wavelengths and the light detector measures the light absorbed or reflected over multiple cardiac cycles to generate one or more signals indicative of a volume of blood present in the renal artery. A metric can be derived from the one or more signals, as described above, and monitored by the implant device 300.

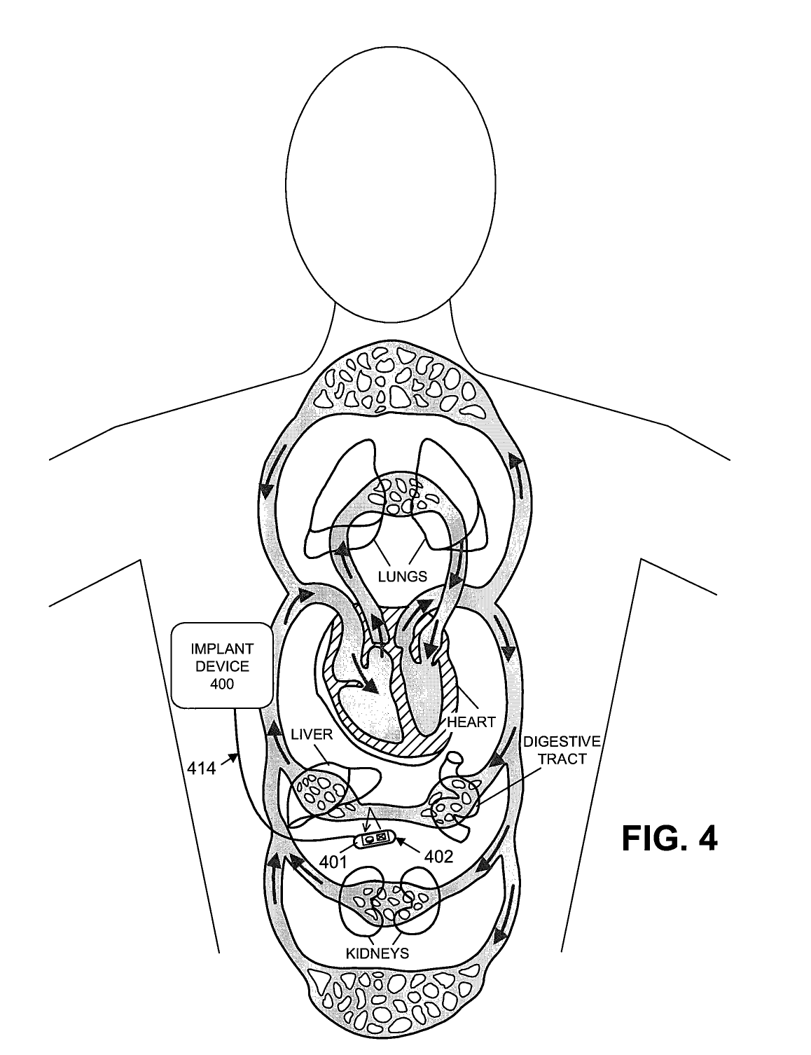

As will be appreciated, systems and methods in accordance with the present invention can be applied to monitor perfusion to multiple different vital and/or non-vital organs. For example, as shown in FIG. 4, an implantable probe 401 including an optical sensor 402 can be positioned so that the optical sensor 402 emits and detects light from an artery carrying blood to the liver. The optical sensor 402 can be positioned to target the hepatic portal vein, for example, or the hepatic artery, and can communicate one or more PPG signals to an implant device 400, for example by way of one or more wires 414. Blood provided by way of the hepatic portal vein is drained from the spleen and gastrointestinal tract. Monitoring blood volume of the hepatic portal vein can provide information about a group of organs including the liver, spleen and organs of the gastrointestinal tract (e.g. the pancreas). Deviation of a derived metric from an established baseline (and range of normal variation) may be indicative of dysfunction and/or failure of one or more organs from the group of organs. As will be appreciated by one of ordinary skill in the arts upon reflecting on the teachings provided herein, systems and methods in accordance with the present invention can be applied with an understanding of anatomy to monitor other vital organs such as the lungs, or non-vital organs such as the spleen, by monitoring blood volume provided to or received from such organs. Embodiments of the present invention are not intended to be limited to systems and methods that target organs as specifically shown in FIGS. 1-9.

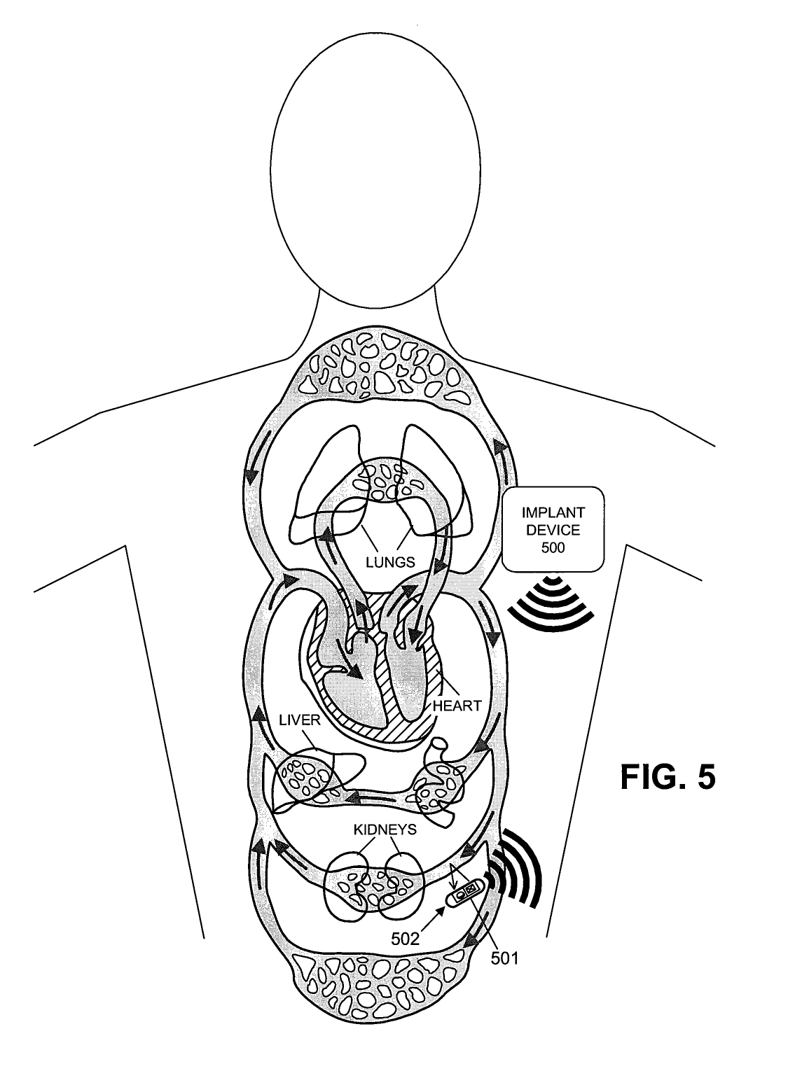

Referring to FIG. 5, an alternative embodiment of a system and method of monitoring blood perfusion to an organ of interest in accordance with the present invention is shown. The system comprises an optical sensor 502 housed in an implantable probe 501, the optical sensor 502 and implantable probe 501 generally resembling those of FIGS. 3A-3C. However, the optical sensor 502 is operably connected with an implant device 500 by a wireless connection. For example, the optical sensor 502 can be operably connected with the implant device 500 using radio signals, a wireless sensor network such as Bluetooth™ or ultra-wideband (UWB), or alternatively using some other wireless implementation. Alternatively, the optical sensor 502 can be operably connected with the implant device 500 using the body as a communication bus, for example as described in U.S. Pat. No. 4,987,897 to Funke.

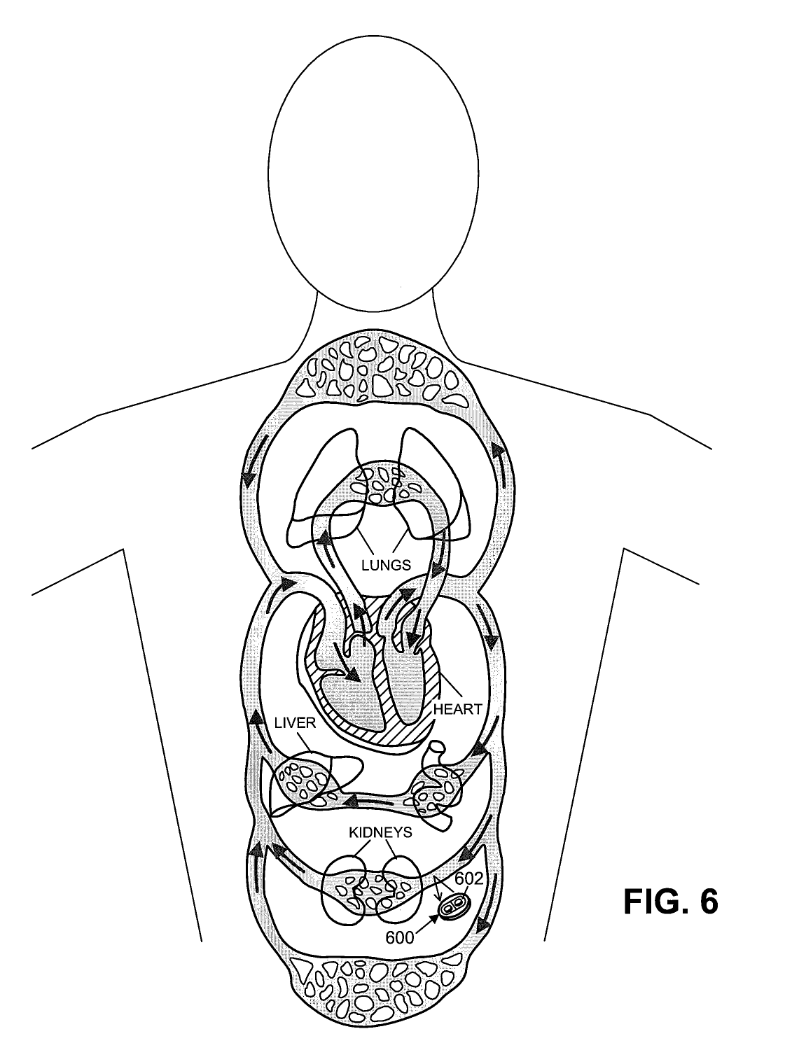

FIG. 6 is a diagram of a further embodiment of a system and method of monitoring blood perfusion to an organ of interest in accordance with the present invention comprising an optical sensor 602 housed or integrally formed with an implant device 600, the implant device 600 positioned so that the optical sensor 502 can measure blood volume at a target site. The implant device 600 includes an energy source to power the optical sensor 602 and circuitry to receive one or more signals from the optical sensor 602.

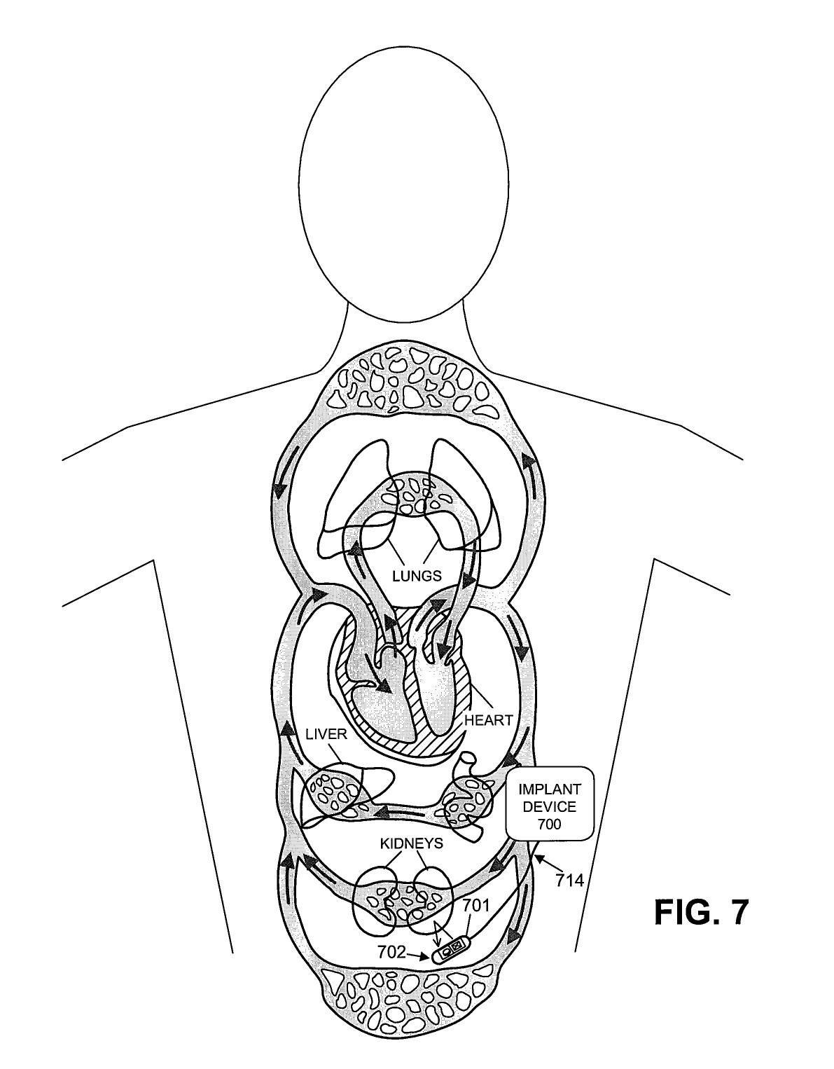

While optical sensors have been described and illustrated herein as being directed to measuring blood within structures providing blood to the organ of interest (e.g. arteries, capillary beds), in other embodiments an optical sensor can be positioned to measure blood received from the organ of interest or blood volume accumulated in the organ of interest. Referring to FIG. 7, a further embodiment of a system and method of monitoring blood perfusion to an organ of interest in accordance with the present invention is shown. The system comprises an optical sensor 702 housed in an implantable probe 701, the optical sensor 702 being positioned to measure blood volume of the organ itself. As above, a single or multi-wavelength light source is directed at the organ and the light detected by a light detector generates a PPG signal(s). A metric is derived from the PPG signal(s) using spectrum analysis, rather than morphology. A “color” of the organ can indicate a volume of blood. For example, a kidney may turn purpler as compared with an established baseline as blood pools in the kidney, whereas the kidney may turn pale as compared with the baseline as blood drains from the kidney. In an embodiment of a system and method of monitoring for tumor growth in accordance with the present invention, an optical sensor can be positioned to measure blood volume in the organ, as shown in FIG. 7. Further, an increase of blood volume in the organ can indicate a recurrence of tumor growth. As above, such systems and methods can be useful for early detection and treatment. For example, where positioned at a pancreas or lung, early detection of tumor growth can greatly improve chances of survival.

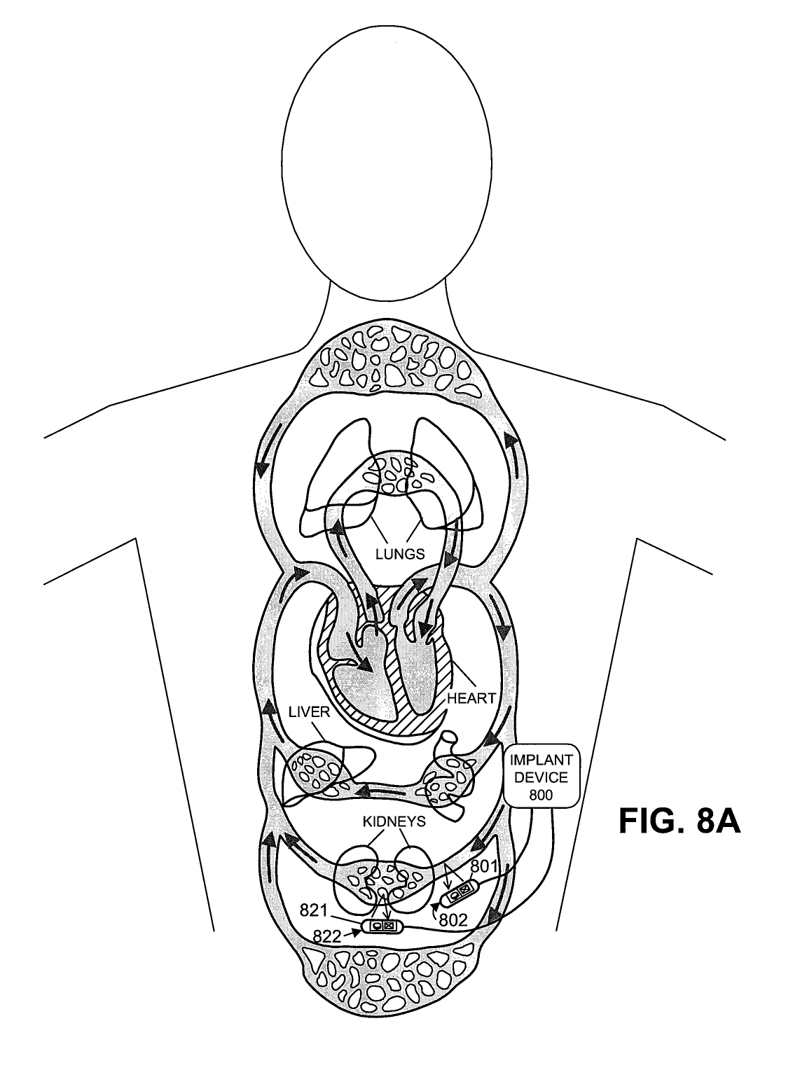

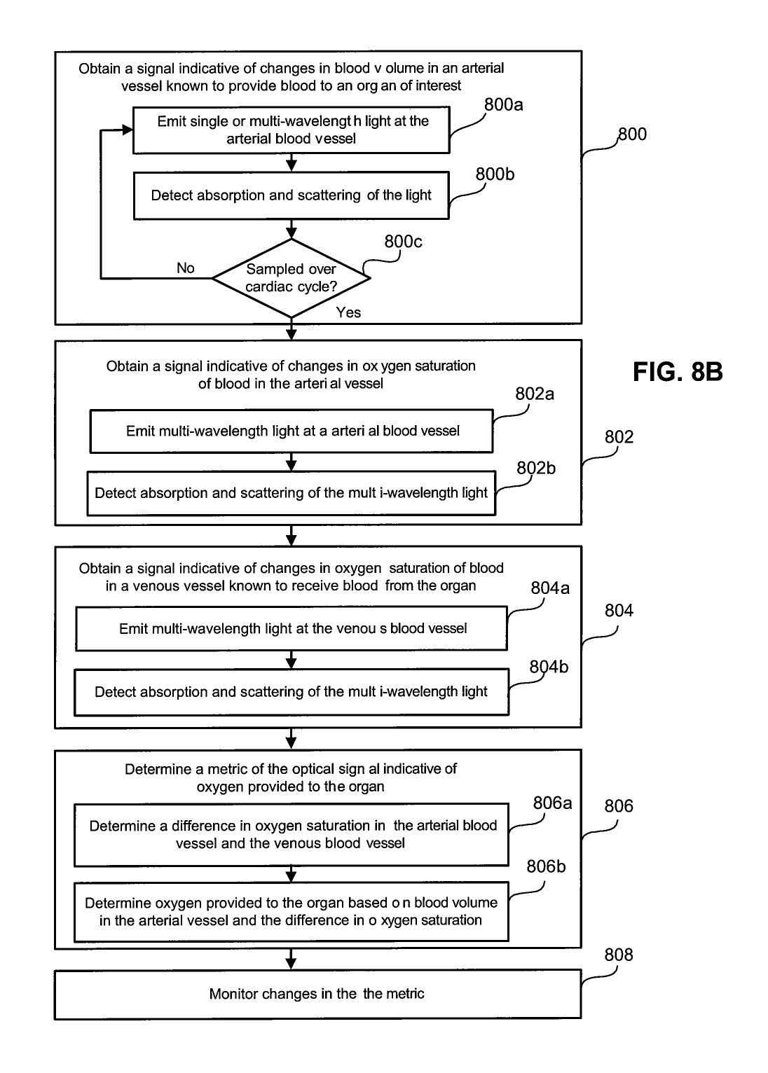

Referring to FIGS. 8A, in other embodiments of systems and methods of monitoring blood perfusion to an organ of interest in accordance with the present invention, a first optical sensor 802 can be positioned to measure oxygen content of blood and/or volume of blood provided to the organ of interest and a second optical sensor 822 can be positioned to measure oxygen content and/or volume of blood leaving the organ of interest. The optical sensors 802, 822 can be housed in corresponding implantable probes 801, 821, operably connected with an implant device 800. The organ can be monitored for changes in total oxygen delivery to the organ, which total oxygen delivery is determined based on blood volume and/or changes in the oxygen saturation of blood provided to the organ (peripheral oxygen saturation, SpO₂, as measured using a pulse oximeter) and the blood volume and/or the oxygen saturation of blood leaving the organ (venous oxygen saturation, SvO₂).

Referring to the flowchart of FIG. 8B, a signal (e.g. a PPG signal) indicative of changes in blood volume provided to the organ (Step 800) can be obtained by emitting single or multi-wavelength pulses of light from a light source of the first optical sensor 802 at an arterial blood vessel known to provide blood to the organ (Step 800a). The absorption and scattering of the emitted light is detected using a light detector of the first optical sensor 802 (Step 800b). The signal is captured over the length of at least one cardiac cycle (Step 800c). Blood volume can be determined based on a PPG generated from the signal. In an embodiment the blood volume is calculated as an area under the curve of the at least one cardiac cycle. One or more signals indicative of oxygen saturation of blood provided to the organ (Step 802) can be obtained by emitting multi-wavelength light from the light source of the first optical sensor 802 at the arterial blood vessel (Step 802a) and detecting the absorption and scattering of the multi-wavelength light using the light detector of the at least one optical sensor 802 (Step 802b). One or more signals indicative of oxygen saturation of blood leaving the organ (Step 804) can be obtained by emitting multi-wavelength light from a light source of a second optical sensor 822 at a venous blood vessel (Step 804a) and detecting the absorption and scattering of the multi-wavelength light using a light detector of the second optical sensor 822 (Step 804b).

A metric indicative of total oxygen delivered to the organ of interest can be determined based on the blood volume provided to the organ, and a difference in oxygen saturation of the blood entering and leaving the organ (Step 806). To generate the metric, the difference in oxygen saturation in the arterial blood vessel and the venous blood vessel is calculated (Step 806a). The total oxygen delivery to the organ can then be calculated as the product of blood volume received by the organ and the difference in oxygen saturation entering and leaving the organ (Step 806c). The metric is monitored for changes that may indicate dysfunction and/or failure in the organ. For example, as shown the optical sensors 802, 822 are positioned to measure total oxygen delivery to a kidney. A decrease in oxygen removed from the blood by the kidney per unit volume of flow and a generally consistent blood volume may indicate anemia. A decrease in oxygen removed from the blood by the kidney per unit volume of flow, coupled with a drop in blood volume may indicate a more serious condition, such as organ failure.

In additional to using the above described optical sensors to measure levels of blood oxygen saturation, such optical sensors can also be used to measure levels of hematocrit, which refers to the percentage of packed red blood cells in a volume of whole blood. Various techniques are known for determining hematocrit based on scattered light. For example, light of about 500 nm and light of about 800 nm can be directed at a blood sample, and an algorithm can be used to calculate hematocrit based on the intensities of detected scattered light. In another technique, a pair of spatially separated light detectors can be used to detect reflected infra red (IR) light, e.g., of 805 nm. The intensity of the IR light detected by the light detector that is nearer to the IR light source is referred to as IRnear, and the intensity of the IR light detected by the light detector farther from the IR light source is referred to as IRfar. As described in article by Bornzin et al., entitled “Measuring Oxygen Saturation and Hematocrit Using a Fiberoptic Catheter”, IEEE/9th Annual Conf. of the Eng. & Biol. Soc. (1997), which is incorporated herein by reference, the ratio: R=IRnear/IRfar is directly related to the level of hematocrit, but independent of oxygen saturation because 805 nm is an isobestic wavelength. To implement this technique using the optical sensor of FIGS. 3B and 3C, a second measurement light detector can be added, with the second measurement light detector being further from (or closer to) the light source(s) than the other measurement light detector. This second measurement light detector can have its own corresponding analog signal processing block and ND converter, or such circuitry can be shared (e.g., multiplexed) with the other measurement light detector. In specific embodiments of the present invention, the second measurement light detector is placed within the optical sensor housing, thereby enabling levels of hematocrit to be measured without the need for relatively large fiber optic guides, use of which was taught in the above mentioned Bornzin et al. article. For example, referring back to FIGS. 3B and 3C, such second measurement light detector can be located farther from the optical wall 360 than the light detector 306 shown.

An alternative metric indicative of total oxygen delivered to the organ of interest can be determined based on the blood volume provided to the organ, and a difference in the level of hematocrit of the blood entering and leaving the organ. To generate the metric, the level of hematocrit in the arterial blood vessel and the venous blood vessel is calculated. The total oxygen delivery to the organ can then be calculated as the product of blood volume received by the organ and the change in level of hematocrit entering and leaving the organ. The metric is monitored for changes that may indicate dysfunction and/or failure in the organ.

Systems and methods have thus far been described as being directed at monitoring dysfunction and/or failure of the targeted organ. However, embodiments of systems and methods in accordance with the present invention can also be applied to measure blood perfusion to an organ of interest in order to monitor for specific systemic dysfunction. For example, sepsis is a serious medical condition characterized by a whole-body inflammatory state (called a systemic inflammatory response syndrome). Sepsis can lead to septic shock, multiple organ dysfunction syndrome and death. Organ dysfunction results from sepsis-induced hypotension and diffuse intravascular coagulation, among other things. Sepsis can be treated with intravenous fluids and antibiotics, as well as other possible measures, such as artificial ventilation and dialysis. However, a problem in the adequate management of septic patients has been the delay in administering therapy after sepsis has been recognized. Published studies have demonstrated that for every hour delay in the administration of appropriate antibiotic therapy there is an associated 7% rise in mortality.

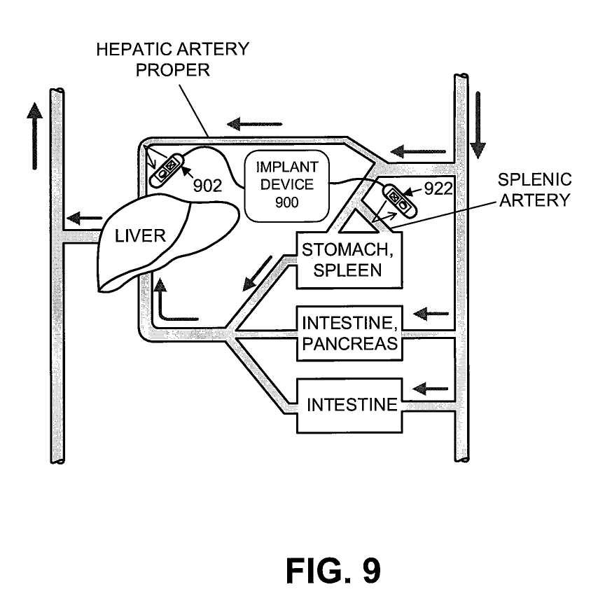

Referring to FIG. 9, an embodiment of a system and method of monitoring for sepsis in accordance with the present invention is shown. The system comprises a first optical sensor 902 housed in a first implantable probe 901 and targeting structures providing blood to a vital organ of interest, and a second optical sensor 922 housed in a second implantable probe 921 and targeting structures providing blood to a non-vital organ of interest. As shown in FIG. 9, the first optical sensor 902 is positioned to target the hepatic artery proper which supplies about 25% of the liver's blood supply, and the second optical sensor 922 is positioned to target the splenic artery which supplies blood to the spleen. Alternatively, the optical sensors 902, 922 can be positioned to measure blood volume of the organs themselves, for example using spectrum analysis as described above. The first optical sensor 902 and second optical sensor 922 can be operably connected with an implant device 900, for example by wires 914, 934 or by way of wireless communication. Alternatively, one common or two separate optical sensors can be physically associated with the implant device, and single or multi-wavelength light can be directed to the two organs by way of fiber optic guides. Alternatively, the optical sensors 902, 922 can communicate with a common device external to the body.

Blood perfusion to the organs can be measured using any of the methods previously described. A metric can be derived from the measurement of blood flow to the non-vital organ relative to the measurement of blood flow to the vital organ. It has been observed that the circulatory system responds to pathogenic microorganisms or their toxins (i.e. sepsis) by increasing blood flow to vital organs and reducing blood flow to non-vital organs. The metric can be monitored for deviations from a baseline indicative of such a systemic response which may be associated with sepsis. Timely detection of the possible onset of sepsis using systems and methods in accordance with the present invention can potentially reduce mortality rates. Further, current practice is to directly prescribe broad spectrum antibiotics to the patient. Timely detection of the possible onset of sepsis using systems and methods in accordance with the present invention can potentially enable more targeted treatment by increasing a window of time for diagnosis so that techniques such as molecular diagnostics can be applied to identify the causative microbe, thereby enabling the more targeted treatment.

Optical sensors and/or implant devices described herein can be positioned to target structures of interest and maintained in position by techniques including suturing, stapling, adhesion, and the like. Alternatively, the optical sensors and/or implant devices can include features such as serrations, barbs, pigtails, spring leaf structures, or other structures to resist migration from the original implantation site. The features or techniques used can be selected based on the tissue surrounding the optical sensors and/or implant devices. For example, small closely space barbs may be more suitable for use at sites where tissues comprises thin fibers.

Optical sensors and/or implant devices described herein can optionally communicate information to a device outside of the patient. For example, a patient can have a monitoring station at the patient's home that communicates wirelessly with the implant device to receive information from the implant device including measurements that can then be communicated remotely to a physician. Alternatively, the implant device can provide a “pass” or “fail” signal to the monitoring station that can signal when a patient should get further diagnostic tests and/or medical treatment. The implant device can communicate actively with the monitoring station, or the implant device can be a passive device that communicates with the monitoring station by telemetry when in communicative proximity. The optical sensors and/or implant device can be powered by any known energy source. Optionally, the energy source can be rechargeable by devices external to the patient. For example, a recharging station can be incorporated into a mat that the patient sleeps on so that the energy source recharges overnight. One of ordinary skill in the art will appreciate the myriad different ways with which the optical sensors and/or implant devices can be powered and can communicate the target organ's health.

While optical sensors and/or implant devices have been described herein as providing a diagnostic tool to monitor organ dysfunction and/or failure and communicate the results to an external computer or physician, in embodiments where the implant device can perform multiple different functions, the implant device can apply treatment or work cooperatively with other devices within the body to correct a perceived dysfunction. For example, neurostimulation has been demonstrated as a technique capable of modifying blood flow. If the implant device monitoring an organ detects a deficiency of oxygen provided to the organ, the implant device can instruct a neurostimulation device to provide increased blood flow to the organ. Conversely, if the implant device detects what it perceives to be a potential tumor, the implant device can instruct the neurostimulation device to shut off blood circulation to the organ as a temporary care measure (such a device would target a non-vital organ or an organ that function in pairs).

While the embodiments of the present invention have been described above as using optical sensors, in many of the above described embodiments alternative types of sensors can be used in place of such optical sensors to obtain the signal(s) indicative of blood being provided to the organ of interest, blood being received from the organ of interest, and/or blood present in the organ of interest. Accordingly, such alternative sensors can be used to monitor changes in an amount of blood being provided to the organ of interest, an amount of blood being received from the organ of interest, and/or an amount of blood present in the organ of interest, based on changes in the obtained signal(s).

For example, impedance sensors can be used to obtain impedance plethysmography signals (IPGs), where such impedance sensors include at least two electrodes, and may also include the circuitry (e.g., circuitry 1094 discussed below) that is used to determine the impedance between at least two electrodes. One or more such electrodes can be, e.g., electrodes located on leads, subcutaneously or otherwise implanted, but are not limited thereto. It is also possible that one such electrode be a conductive housing of an implant device.

In still other embodiments, such signal(s) can be output by a sensor including a piezo-electric diaphragm. Alternative sensors that can be used to obtain the signal(s) of interest, include, but are not limited to, a close range microphone, a sensor including a small mass on the end of a piezo bending beam with the mass located on the surface of a small artery, a transmission mode infrared motion sensor sensing across the surface of a small artery, or a MEMS accelerometer located on the surface of a small artery. Such alternative sensors can be located, e.g., on the tip of a short lead connected to a device that is subcutaneously implanted. The alternative implanted sensors can be implanted, e.g., extravascularly at the various different locations described above with reference to the various FIGS. In certain embodiments, such alternative sensors are not implanted, but rather are located against a patient's skin adjacent the location(s) of interest, e.g., adjacent an organ of interest, a blood vessel providing blood to the organ of interest, or a blood vessel providing blood from the organ of interest.

Ultrasound sensors can also be used. With acoustics and ultrasound sensor the idea is similar. When sound (or ultrasound) is emitted into the tissue, the sound (or ultrasound) will reflect back in a predictable pattern, similar to imaging or Doppler. By monitoring changes in the reflected sound (or ultrasound), changes in blood volume can be detected. If Doppler is used, actually measure the blood flow can be determined. Further, if desired, one or more measures indicative of pulse arrival time can be measured (which are indicative of an amount of time between a heart's contraction and when a resulting pulse arrives at the sensor), which are useful for monitoring of changes in blood flow.

Alternative embodiments of the present invention encompass the use of such alternative sensors. In other words, embodiments of the present invention are not limited to using optical sensors.

### Exemplary Implant Device

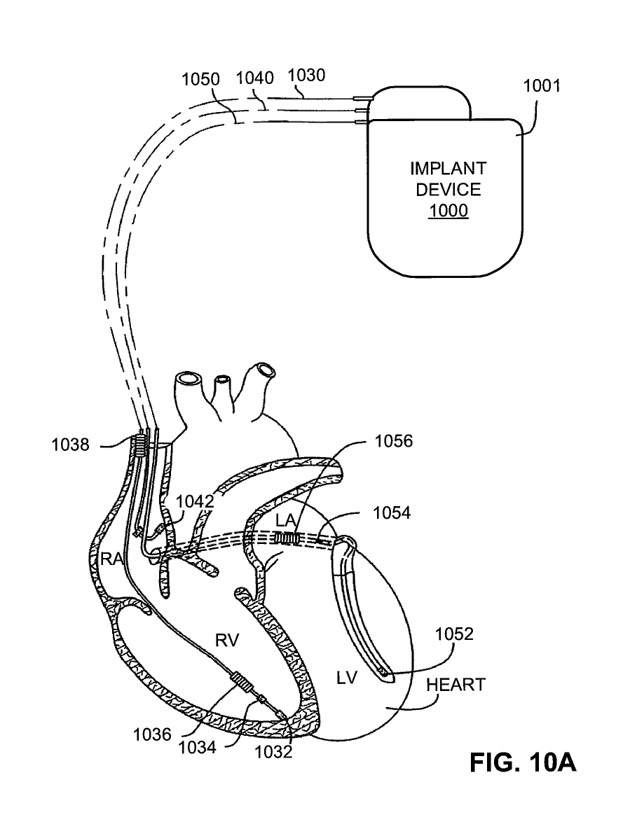

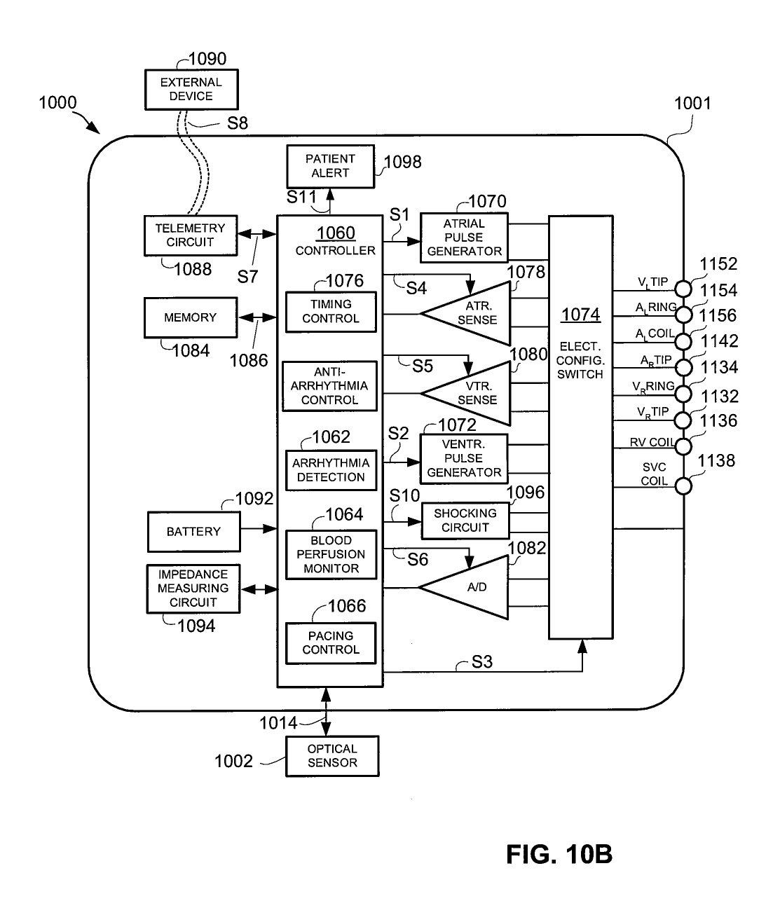

FIG. 10A illustrates an exemplary implant device with which optical sensors and methods of monitoring blood perfusion in accordance with embodiments of the present invention can be used. The implant device 1000 is shown comprising an implantable stimulation device, which can be a pacing device and/or an implantable cardioverter defibrillator. The implant device 1000 is shown as being in electrical communication with a patient's heart by way of three leads 1030, 1040, and 1050, which can be suitable for delivering multi-chamber stimulation and shock therapy.

To sense atrial cardiac signals and to provide right atrial chamber stimulation therapy, the device 1000 is coupled to an implantable right atrial lead 1040 having at least an atrial tip electrode 1042, which typically is implanted in the patient's right atrial appendage. To sense left atrial and ventricular cardiac signals and to provide left-chamber pacing therapy, the device 1000 is coupled to a “coronary sinus” lead 1050 designed for placement in the “coronary sinus region” via the coronary sinus for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, an exemplary coronary sinus lead 1050 is designed to receive atrial and ventricular cardiac signals and to deliver left ventricular pacing therapy using at least a left ventricular tip electrode 1052, left atrial pacing therapy using at least a left atrial ring electrode 1054, and shocking therapy using at least a left atrial coil electrode 1056.

The device 1000 is also shown in electrical communication with the patient's heart by way of an implantable right ventricular lead 1030 having, in this embodiment, a right ventricular tip electrode 1032, a right ventricular ring electrode 1034, a right ventricular (RV) coil electrode 1036, and an SVC coil electrode 1038. Typically, the right ventricular lead 1030 is transvenously inserted into the heart so as to place the right ventricular tip electrode 1032 in the right ventricular apex so that the RV coil electrode 1036 will be positioned in the right ventricle and the SVC coil electrode 1038 will be positioned in the superior vena cava. Accordingly, the right ventricular lead 1030 is capable of receiving cardiac signals and delivering stimulation in the form of pacing and shock therapy to the right ventricle.

FIG. 10B will now be used to provide some exemplary details of the components of the implant device 1000. Referring now to FIG. 10B, each of the above implant device 1000, and alternative versions thereof, can include a microcontroller 1060. As is well known in the art, the microcontroller 1060 typically includes a microprocessor, or equivalent control circuitry, and can further include RAM or ROM memory, logic and timing circuitry, state machine circuitry, and I/O circuitry. Typically, the microcontroller 1060 includes the ability to process or monitor input signals (data) as controlled by a program code stored in a designated block of memory. The details of the design of the microcontroller 1060 are not critical to the present invention. Rather, any suitable microcontroller 1060 can be used to carry out the functions described herein. The use of microprocessor-based control circuits for performing timing and data analysis functions are well known in the art. In specific embodiments of the present invention, the microcontroller 1060 performs some or all of the steps associated with monitoring blood perfusion to an organ of interest within a patient, monitoring blood volume and tumor growth in an organ, and/or monitoring for sepsis.

Representative types of control circuitry that may be used with the invention include the microprocessor-based control system of U.S. Pat. No. 4,940,052 (Mann et. al.) and the state-machines of U.S. Pat. No. 4,712,555 (Sholder) and U.S. Pat. No. 4,944,298 (Sholder). For a more detailed description of the various timing intervals used within the pacing device and their inter-relationship, see U.S. Pat. No. 4,788,980 (Mann et. al.). The '052, '555, '298 and '980 patents are incorporated herein by reference.

Depending on implementation, the implant device 1000 can be capable of treating both fast and slow arrhythmias with stimulation therapy, including pacing, cardioversion and defibrillation stimulation. While a particular multi-chamber device is shown, this is for illustration purposes only, and one of skill in the art could readily duplicate, eliminate or disable the appropriate circuitry in any desired combination to provide a device capable of treating the appropriate chamber(s) with pacing, cardioversion and defibrillation stimulation. For example, where the implantable device is a monitor that does not provide any therapy, it is clear that many of the blocks shown may be eliminated.

The housing 1001, shown schematically in FIG. 10B, is often referred to as the “can”, “case” or “case electrode” and may be programmably selected to act as the return electrode for all “unipolar” modes. The housing 1001 may further be used as a return electrode alone or in combination with one or more of the coil electrodes, 1036 and 1038, 1056, for shocking purposes. The housing 1001 can further include a connector (not shown) having a plurality of terminals, 1132, 1134, 1136, 1138, 1142, 1152, 1154, and 1156 (shown schematically and, for convenience, the names of the electrodes to which they are connected are shown next to the terminals). As such, to achieve right atrial sensing and pacing, the connector includes at least a right atrial tip terminal (A_{R} TIP) 1142 adapted for connection to the atrial tip electrode 1042.

To achieve left atrial and ventricular sensing, pacing and shocking, the connector includes at least a left ventricular tip terminal (V_{L} TIP) 1152, a left atrial ring terminal (A_{L} RING) 1154, and a left atrial shocking terminal (A_{L} COIL) 1156, which are adapted for connection to the left ventricular ring electrode 1052, the left atrial tip electrode 1054, and the left atrial coil electrode 1056, respectively.

To support right ventricle sensing, pacing and shocking, the connector further includes a right ventricular tip terminal (V_{R} TIP) 1132, a right ventricular ring terminal (V_{R} RING) 1134, a right ventricular shocking terminal (R_{V} COIL) 1136, and an SVC shocking terminal (SVC COIL) 1138, which are adapted for connection to the right ventricular tip electrode 1032, right ventricular ring electrode 1034, the RV coil electrode 1036, and the SVC coil electrode 1038, respectively.

An atrial pulse generator 1070 and a ventricular pulse generator 1072 generate pacing stimulation pulses for delivery by the right atrial lead 1040, the right ventricular lead 1030, and/or the coronary sinus lead 1050 via an electrode configuration switch 1074. It is understood that in order to provide stimulation therapy in each of the four chambers of the heart, the atrial and ventricular pulse generators, 1070 and 1072, may include dedicated, independent pulse generators, multiplexed pulse generators, or shared pulse generators. The pulse generators, 1070 and 1072, are controlled by the microcontroller 1060 via appropriate control signals, S1 and S2 respectively, to trigger or inhibit the stimulation pulses.

The microcontroller 1060 further includes timing control circuitry 1076 which is used to control pacing parameters (e.g., the timing of stimulation pulses) as well as to keep track of the timing of refractory periods, noise detection windows, evoked response windows, alert intervals, marker channel timing, etc., which is well known in the art. Examples of pacing parameters include, but are not limited to, atrio-ventricular delay, interventricular delay and interatrial delay.

The switch bank 1074 includes a plurality of switches for connecting the desired electrodes to the appropriate I/O circuits, thereby providing complete electrode programmability. Accordingly, the switch 1074, in response to a control signal S3 from the microcontroller 1060, determines the polarity of the stimulation pulses (e.g., unipolar, bipolar, etc.) by selectively closing the appropriate combination of switches (not shown) as is known in the art.

Atrial sensing circuits 1078 and ventricular sensing circuits 1080 may also be selectively coupled to the right atrial lead 1040, coronary sinus lead 1050, and the right ventricular lead 1030, through the switch 1074 for detecting the presence of cardiac activity in each of the four chambers of the heart. Accordingly, the atrial (ATR. SENSE) and ventricular (VTR. SENSE) sensing circuits, 1078 and 1080, may include dedicated sense amplifiers, multiplexed amplifiers, or shared amplifiers. The switch 1074 determines the “sensing polarity” of the cardiac signal by selectively closing the appropriate switches, as is also known in the art. In this way, the clinician may program the sensing polarity independent of the stimulation polarity.

Each sensing circuit, 1078 and 1080, preferably employs one or more low power, precision amplifiers with programmable gain and/or automatic gain control, bandpass filtering, and a threshold detection circuit, as known in the art, to selectively sense the cardiac signal of interest. The automatic gain control enables the device 1000 to deal effectively with the difficult problem of sensing the low amplitude signal characteristics of atrial or ventricular fibrillation. Such sensing circuits, 1078 and 1080, can be used to determine cardiac performance values used in the present invention. Alternatively, an automatic sensitivity control circuit may be used to effectively deal with signals of varying amplitude.

The outputs of the atrial and ventricular sensing circuits, 1078 and 1080, are connected to the microcontroller 1060 which, in turn, are able to trigger or inhibit the atrial and ventricular pulse generators, 1070 and 1072, respectively, in a demand fashion in response to the absence or presence of cardiac activity, in the appropriate chambers of the heart. The sensing circuits, 1078 and 1080, in turn, receive control signals over signal lines, S4 and S5, from the microcontroller 1060 for purposes of measuring cardiac performance at appropriate times, and for controlling the gain, threshold, polarization charge removal circuitry (not shown), and timing of any blocking circuitry (not shown) coupled to the inputs of the sensing circuits, 1078 and 1080.

For arrhythmia detection, the device 1000 includes an arrhythmia detector 1062 that utilizes the atrial and ventricular sensing circuits, 1078 and 1080, to sense cardiac signals to determine whether a rhythm is physiologic or pathologic. The timing intervals between sensed events (e.g., P-waves, R-waves, and depolarization signals associated with fibrillation) can be classified by the microcontroller 1060 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to assist with determining the type of remedial therapy that is needed (e.g., bradycardia pacing, anti-tachycardia pacing, cardioversion shocks or defibrillation shocks, collectively referred to as “tiered therapy”). Additionally, the arrhythmia detector 1062 can perform arrhythmia discrimination, e.g., using measures of arterial blood pressure determined in accordance with embodiments of the present invention. Exemplary details of such arrhythmia discrimination, including tachyarrhythmia classification, are discussed above. The arrhythmia detector 1062 can be implemented within the microcontroller 1060, as shown in FIG. 10B. Thus, this detector 1062 can be implemented by software, firmware, or combinations thereof. It is also possible that all, or portions, of the arrhythmia detector 1062 can be implemented using hardware. Further, it is also possible that all, or portions, of the arrhythmia detector 1062 can be implemented separate from the microcontroller 1060.

In accordance with embodiments of the present invention, the implant device 1000 includes a blood perfusion monitor 1064, which can monitor blood perfusion to an organ of interest and/or blood volume of an organ of interest using the techniques described above with reference to FIGS. 2A-8B. The blood perfusion monitor 1064 can be implemented within the microcontroller 1060, as shown in FIG. 10B, and can be implemented using software, firmware, or combinations thereof. It is also possible for all, or portions, of the blood perfusion monitor 1064 to be implemented using hardware. Further, it is also possible for all, or portions, of the blood perfusion monitor 1064 to be implemented separate from the microcontroller 1060. The microcontroller 1060 can receive one or more PPG signals from an optical sensor 1002 positioned to monitor a structure associated with an organ of interest. As noted above, the one or more PPG signals can be received by way of one or more wires 1014, one or more fiber optic guide, or wireless communication. Further, the one or more PPG signals can be received by way of the telemetry circuit 1088 described below. Alternatively, or additionally, the monitor 1064 (or a separate monitor) can monitor both a vital organ and a non-vital organ for relative changes in blood flow that may be indicative of sepsis using the techniques described above with reference to FIG. 9. Alternatively, or additionally, the monitor 1064 (or a separate monitor) can monitor blood volume in an organ to identify dysfunctions such as tumor grown using the techniques described above with reference to FIG. 7.

The implantable device 1000 can also include a pacing controller 1066, which can adjust a pacing rate and/or pacing intervals. The pacing controller 1066 can be implemented within the microcontroller 1060, as shown in FIG. 10B. Thus, the pacing controller 1066 can be implemented by software, firmware, or combinations thereof. It is also possible that all, or portions, of the pacing controller 1066 can be implemented using hardware. Further, it is also possible that all, or portions, of the pacing controller 1066 can be implemented separate from the microcontroller 1060.

Still referring to FIG. 10B, cardiac signals are also applied to the inputs of an analog-to-digital (A/D) data acquisition system 1082. The data acquisition system 1082 is configured to acquire IEGM and/or ECG signals, convert the raw analog data into a digital signal, and store the digital signals for later processing and/or telemetric transmission to an external device 1090. The data acquisition system 1082 can be coupled to the right atrial lead 1040, the coronary sinus lead 1050, and the right ventricular lead 1030 through the switch 1074 to sample cardiac signals across any pair of desired electrodes.

The data acquisition system 1082 can be coupled to the microcontroller 1060, or other detection circuitry, for detecting an evoked response from the heart in response to an applied stimulus, thereby aiding in the detection of “capture”. Capture occurs when an electrical stimulus applied to the heart is of sufficient energy to depolarize the cardiac tissue, thereby causing the heart muscle to contract. The microcontroller 1060 detects a depolarization signal during a window following a stimulation pulse, the presence of which indicates that capture has occurred. The microcontroller 1060 enables capture detection by triggering the ventricular pulse generator 1072 to generate a stimulation pulse, starting a capture detection window using the timing control circuitry 1076 within the microcontroller 1060, and enabling the data acquisition system 1082 via control signal S6 to sample the cardiac signal that falls in the capture detection window and, based on the amplitude, determines if capture has occurred.

The implementation of capture detection circuitry and algorithms are well known. See for example, U.S. Pat. No. 4,729,376 (Decote, Jr.); U.S. Pat. No. 4,708,142 (Decote, Jr.); U.S. Pat. No. 4,686,988 (Sholder); U.S. Pat. No. 4,969,467 (Callaghan et. al.); and U.S. Pat. No. 5,350,410 (Mann et. al.), which patents are hereby incorporated herein by reference. The type of capture detection system used is not critical to the present invention.

The microcontroller 1060 is further coupled to the memory 1084 by a suitable data/address bus 1086, wherein the programmable operating parameters used by the microcontroller 1060 are stored and modified, as required, in order to customize the operation of the implantable device 1000 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart within each respective tier of therapy. The memory 1084 can also store data about blood perfusion and/or blood volume in an organ of interest.

The operating parameters of the implantable device 1000 may be non-invasively programmed into the memory 1084 through a telemetry circuit 1088 in telemetric communication with an external device 1090, such as a programmer, transtelephonic transceiver, or a diagnostic system analyzer. The telemetry circuit 1088 can be activated by the microcontroller 1060 by a control signal S7. The telemetry circuit 1088 advantageously allows intracardiac electrograms and status information relating to the operation of the device 1000 (as contained in the microcontroller 1060 or memory 1084) to be sent to the external device 1090 through an established communication link S8. The telemetry circuit 1088 can also be use to transmit arterial blood pressure data to the external device 1090. Optionally, the implant device 1000 can further include a patient alert 1098 that can indicate heart, and/or other organ dysfunction. The patient alert 1098 receives a signal S11 from the controller 1060 when predefined conditions are met.

For examples of telemetry devices, see U.S. Pat. No. 4,809,697, entitled “Interactive Programming and Diagnostic System for use with Implantable Pacemaker” (Causey, Ill et al.); U.S. Pat. No. 4,944,299, entitled “High Speed Digital Telemetry System for Implantable Device” (Silvian); and U.S. Pat. No. 6,275,734 entitled “Efficient Generation of Sensing Signals in an Implantable Medical Device such as a Pacemaker or ICD” (McClure et al.), which patents are hereby incorporated herein by reference.

The implantable device 1000 additionally includes a battery 1092 which provides operating power to all of the circuits shown in FIG. 10B. If the implantable device 1000 also employs shocking therapy, the battery 1092 should be capable of operating at low current drains for long periods of time, and then be capable of providing high-current pulses (for capacitor charging) when the patient requires a shock pulse. The battery 1092 should also have a predictable discharge characteristic so that elective replacement time can be detected.

The implantable device 1000 can also include a magnet detection circuitry (not shown), coupled to the microcontroller 1060. It is the purpose of the magnet detection circuitry to detect when a magnet is placed over the implantable device 1000, which magnet may be used by a clinician to perform various test functions of the implantable device 1000 and/or to signal the microcontroller 1060 that the external programmer 1090 is in place to receive or transmit data to the microcontroller 1060 through the telemetry circuits 1088.

As further shown in FIG. 10B, the implant device 1000 is also shown as having an impedance measuring circuit 1094 which is enabled by the microcontroller 1060 via a control signal S9. The known uses for an impedance measuring circuit 1094 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds and heart failure condition; detecting when the device has been implanted; measuring stroke volume; and detecting the opening of heart valves, etc. The impedance measuring circuit 1094 is advantageously coupled to the switch 1074 so that any desired electrode may be used. The impedance measuring circuit 1094 is not critical to the present invention and is shown only for completeness.

In the case where the implant device 1000 is also intended to operate as an implantable cardioverter/defibrillator (ICD) device, it should detect the occurrence of an arrhythmia, and automatically apply an appropriate electrical shock therapy to the heart aimed at terminating the detected arrhythmia. To this end, the microcontroller 1060 further controls a shocking circuit 1096 by way of a control signal S10. The shocking circuit 1096 generates shocking pulses of low (up to 0.5 Joules), moderate (0.5-10 Joules), or high energy (11 to 40 Joules), as controlled by the microcontroller 1060. Such shocking pulses are applied to the patient's heart through at least two shocking electrodes, and as shown in this embodiment, selected from the left atrial coil electrode 1056, the RV coil electrode 1036, and/or the SVC coil electrode 1038. As noted above, the housing 1001 may act as an active electrode in combination with the RV electrode 1036, or as part of a split electrical vector using the SVC coil electrode 1038 or the left atrial coil electrode 1056 (i.e., using the RV electrode as a common electrode).

The above described implantable device 1000 was described as an exemplary pacing device. One or ordinary skill in the art would understand that embodiments of the present invention can be used with alternative types of implantable devices. Accordingly, embodiments of the present invention should not be limited to use only with the above described device.

The present invention has been described above with the aid of functional building blocks illustrating the performance of specified functions and relationships thereof. The boundaries of these functional building blocks have often been arbitrarily defined herein for the convenience of the description. Alternate boundaries can be defined so long as the specified functions and relationships thereof are appropriately performed. Any such alternate boundaries are thus within the scope and spirit of the claimed invention. For example, it would be possible to combine or separate some of the steps shown in the flow diagrams. Further, it may be possible to change the order of some of the steps shown in flow diagrams, without substantially changing the overall events and results. For another example, it is possible to change the boundaries of some of the blocks shown in FIG. 10B.

The previous description of the preferred embodiments is provided to enable any person skilled in the art to make or use the embodiments of the present invention. While the invention has been particularly shown and described with reference to preferred embodiments thereof, it will be understood by those skilled in the art that various changes in form and details may be made therein without departing from the spirit and scope of the invention.

What is claimed is:

1. A method for monitoring an organ of interest within a patient, the method comprising:
(a) using one or more sensors to obtain one or more signals indicative of one or more of blood being provided to the organ of interest, blood being received from the organ of interest, and blood present in the organ of interest; and
(b) monitoring changes in one or more of an amount of blood being provided to the organ of interest, an amount of blood being received from the organ of interest, and an amount of blood present in the organ of interest, based on changes in at least one of the one or more obtained signals.

2. The method of claim 1, wherein each of the one or more sensors comprises an impedance sensor, each impedance sensor including at least two electrodes, and each of the obtained signals comprises an impedance plethysmography signal.

3. The method of claim 1, wherein each of the one or more sensors comprises an optical sensor, each optical sensor including a light source and a light detector, and each of the obtained signals comprises a photoplethysmography signal.

4. The method of claim 1, wherein step (a) includes using said one or more sensors to obtain one or more plethysmography signals indicative of one or more of blood being provided to the organ of interest, blood being received from the organ of interest, and blood present in the organ of interest.

5. The method of claim 1, wherein step (b) includes:
(b.1) determining, from time to time, one or more metrics based on at least one of the one or more obtained signals, wherein the one or more metrics is/are indicative of one or more of blood being provided to the organ of interest, blood being received from the organ of interest, and blood present in the organ of interest; and
(b.2) monitoring changes one or more said determined metrics, over time, to thereby monitor changes in one or more of an amount of blood being provided to the organ of interest, an amount of blood being received from the organ of interest, and an amount of blood present in the organ of interest.

6. The method of claim 5, wherein step (b.2) includes monitoring whether one or more of the amount of blood being provided to the organ of interest, the amount of blood being received from the organ of interest, and the amount of blood present in the organ of interest is increasing, decreasing or staying relatively the same.

7. The method of claim 5, wherein step (b) includes (b.3) comparing a said metric to a corresponding baseline; and further comprising:
(c) triggering an alert and/or therapy if the said metric falls below or rises above the corresponding baseline by at least a specified threshold.

8. The method of claim 5, wherein a said metric determined at step (b.1) is indicative of blood volume of blood vessels known to provide blood to the organ of interest.

9. The method of claim 5, wherein a said metric determine at step (b.1) is indicative of venous oxygen saturation or arterial oxygen saturation.

10. The method of claim 5, wherein:
the organ of interest is a kidney;
step (a) includes using at least one said sensor to obtain a signal indicative of renal blood flow being provided to the kidney; and
step (b) includes monitoring changes in the obtained signal indicative of renal blood flow being provided to the kidney, to thereby monitor changes in the amount of renal blood flow being provided to the kidney.

11. The method of claim 10, wherein step (a) includes using at least one said sensor to obtain a plethysmography signal indicative of changes in blood volume of blood vessels selected from the group consisting of:
glomerular capillaries;
renal arteries; and
renal veins.

12. The method of claim 11, wherein:
step (b) includes
(b.1) determining, from time to time, a metric based the obtained plethysmography signal indicative of renal blood flow being provided to the kidney; and
(b.2) comparing the determined metric to a baseline; and further comprising
(c) detecting kidney disfunction if the metric falls below the baseline by at least a specified threshold.

13. The method of claim 5, wherein a said metric determined at (b.1) is determined based on at least one of:
a peak-to-peak amplitude of one or more of the obtained signals;
an area under the curve of one or more of the obtained signals;
a full width at have max of one or more of the obtained signals; and
a downward slope of after a peak amplitude of one or more of the obtained signals

14. The method of claim 1, wherein:
step (a) comprises obtaining one or more optical signals indicative of absorption and/or scattering of light at different wavelengths caused by the blood present in the organ of interest.

15. The method of claim 1, wherein at least one said sensor is implanted extravascularly within the patient at a location adjacent to the organ of interest or one or more blood vessels that provide blood to or receive blood from the organ of interest.

16. The method of claim 1, wherein at least one said sensor is a non-implanted sensor that is located against the patient's skin at a location adjacent to one or more blood vessels that provide blood to or receive blood from the organ of interest.

17. The method of claim 1, further comprising:
(c) triggering an alert and/or therapy if a monitored change in the one or more of an amount of blood provided to the organ of interest, an amount of blood being received from the organ of interest, and an amount of blood present in the organ of interest is indicative of dysfunction of the organ of interest.

18. The method of claim 1, further comprising:
(c) monitoring for growth of a tumor in the organ of interest based on the monitored changes in one or more of an amount of blood provided to the organ of interest, an amount of blood being received from the organ of interest, and an amount of blood present in the organ of interest; and
(d) triggering an alert if the monitored changes are indicative of tumor growth in the organ of interest.

19. A system for monitoring an organ of interest within a patient, comprising:
one or more sensors configured to obtain one or more signals indicative of one or more of blood being provided to the organ of interest, blood being received from the organ of interest, and blood present in the organ of interest; and
a monitor configured to monitor changes in one or more of an amount of blood being provided to the organ of interest, an amount of blood being received from the organ of interest, and an amount of blood present in the organ of interest, based on changes in at least one of the one or more of the signals obtained by said one or more sensors.

20. The system of claim 19, wherein the monitor is configured to:
determine, from time to time, one or more metrics based on at least one of the one or more obtained signals, wherein the one or more metrics is/are indicative of one or more of blood being provided to the organ of interest, blood being received from the organ of interest, and blood present in the organ of interest; and
monitor changes in the one or more determined metrics, over time, to thereby monitor changes in one or more of an amount of blood provided to the organ of interest, an amount of blood being received from the organ of interest, and an amount of blood present in the organ of interest.

21. The system of claim 20, wherein the monitor is also configured to trigger an alert and/or therapy if a monitored change in one or more said metrics is indicative of dysfunction of the organ of interest.

22. The system of claim 19, wherein each of the one or more sensors comprises an impedance sensor, each impedance sensor including at least two electrodes.

23. The system of claim 19, wherein each of the one or more sensors comprises an optical sensor, each optical sensor including a light source and a light detector.

24. A method for monitoring for sepsis, the method comprising:
(a) using one or more sensors to obtain one or more signals indicative of one or more of blood being provided to a vital organ, blood being received from the vital organ, and blood present in the vital organ;
(b) using one or more further sensors to obtain one or more signals indicative of one or more of blood being provided to a non-vital organ, blood being received from the non-vital organ, and blood present in the non-vital organ;
(c) monitoring for sepsis based on a comparison between the one or more signals obtained at step (a) and the one or more signals obtained at step (b); and
(d) triggering an alert and/or therapy if sepsis is detected.

25. The method of claim 24, wherein step (c) comprises:
(c.1) determining one or more metrics of one or more signals indicative of one or more of blood being provided to the vital organ, blood being received from the vital organ, and blood present in the vital organ;
(c.2) determining one or more metrics of one or more signals indicative of one or more of blood being provided to the non-vital organ, blood being received from the non-vital organ, and blood present in the non-vital organ; and
(c.3) determining one or more metrics indicative of a difference between a said metric determined at (c.1) and a corresponding said metric determined at (c.2); and
(c.4) monitoring for sepsis based on at least one said metric indicative of the difference determined at (c.3).
